(19) United States
(12) Patent Application Publication
(10) Pub. No.: US 2010/0087744 A1
(43) Pub. Date: Apr. 8, 2010

(54) Pulse rate monitor and process for detecting an allergen

(76) Inventor: Florence J. Licata, Vero Beach, FL (US)

Correspondence Address: JOHN RYZNIC, FLORIDA TURBINE TECHNOLOGIES, INC., 1701 MILITARY TRAIL, SUITE 110, JUPITER, FL, 33458-7887, US

(21) Appl. No.: 12/246,745

(22) Filed: Oct. 7, 2008

(51) Int. Cl. A61B 5/02

(52) U.S. Cl. 600/503

(57) ABSTRACT

A wearable pulse rate and rhythm monitoring device that monitors a person's pulse for variables such as pulse rate, pulse rhythm, pulse amplitude and blood pressure versus time when a person is exposed to one or more potential allergens. The person will select a potential allergen, such as corn, from a list stored in the device when the person will be exposed to that particular potential allergen. Selecting a potential allergen will start the device to monitor the pulse for gathering the data and storing the data in the memory for a pre-selected period of time. The stored data is then downloaded into a program that will plot the pulse rate versus real time so that an allergen can be identified. By monitoring the various changes to a person's pulse versus real time instead of taking an average, even slight allergens can be identified.

### FEDERAL RESEARCH STATEMENT

None.

### CROSS-REFERENCE TO RELATED APPLICATIONS

None.

### BACKGROUND OF THE INVENTION

1. Field of the Invention

The present invention relates generally to determining a person's allergic reaction, and more specifically to a device worn by a person that can monitor a person's pulse and store the data in real time in order to detect for an allergic reaction.

2. Description of the Related Art Including Information Disclosed Under 37 CFR 1.97 and 1.98

It is known that some people can have an allergic react to a certain food or environmental stimulant that others would not. A person allergic to certain food, for example, when that food is eaten will have an allergic reaction characterized by a change in their pulse rate and/or rhythm, and sometimes also amplitude.

U.S. Pat. No. 5,897,506 issued to Cohn on Apr. 27, 1999 and entitled PULSE RATE MONITOR FOR ALLERGY DETECTION AND CONTROL—incorporated herein by reference—discloses a pulse rate monitor that is used by a person that will detect a change in the pulse rate due to an allergic reaction in order to determine the allergen. The Cohn invention only monitors the pulse rate of the person, and then only takes an average of the pulse rate over the time period being monitored. In slight allergic reactions, an average reading of the pulse rate will not even show an allergic reaction. Also, some allergic reactions do not change the pulse rate but will change the rhythm or even the amplitude. Thus, some allergic reactions will be missed because the device is only monitoring the pulse rate.

### BRIEF SUMMARY OF THE INVENTION

It is an object of the present invention to detect for an allergic reaction in a person exposed to an allergen.

It is another object of the present, invention to obtain real time data of a pulse from a person exposed to an allergen for use in detecting for an allergic reaction.

It is another object of the present invention to provide for a process in which an allergen can be reintroduced into a person in such small quantities such that the allergic reaction can be eliminated.

The present invention is a device worn by an individual, such as a wrist watch type device, that can monitor the person's pulse rate, rhythm and amplitude and blood pressure while the person is eating or exposed to certain elements that may produce an allergic response, and store the data collected in real time for later use. The data is collected in real time and stored in a memory for a period of time in which an allergic reaction can be detected from the data. The data captured during the monitoring process is then downloaded to a software program that is used to determine if an allergic reaction occurred. The data for each allergen is plotted on a graph representing the pulse rate, rhythm and amplitude and the blood pressure versus time for the predetermined period of time. If an allergic reaction occurred, then the certain food item or environmental stimuli can then be identified from spikes in the graph. A software program uses the gathered data to plot pulse rate, rhythm and other data in real time to spot small changes indicative of an allergic reaction that could not be identified by averaging based data analysis. Thus, a more accurate reading of the data is obtained in order to identify allergens. In real time, it is meant that the actual pulse rate versus time for the entire monitoring period is captured and stored for display on a graph. Thus, the actual pulse rates, pulses rhythms and other data can be plotted on the graph versus time for observation of the individual data points obtained from the pulse and blood pressure monitor.

For example, a person would eat a certain food item such as corn and activate the pulse rate monitor and enter what food item was being consumed into the device. Later, that person could eat another food item such as wheat bread, enter into the device that the food item being consumed was wheat bread, and then activate the device to monitor the various pulse rate and rhythm signals. Later, the data from the two food items is analyzed to determine if and how much of an allergic reaction was caused by that specific allergen.

### BRIEF DESCRIPTION OF THE SEVERAL VIEWS OF THE DRAWINGS

FIG. 1 shows a block diagram schematic representing the elements of the pulse rate monitoring device.

FIG. 2 shows a flow chart of the process for detecting an allergen.

### DETAILED DESCRIPTION OF THE INVENTION

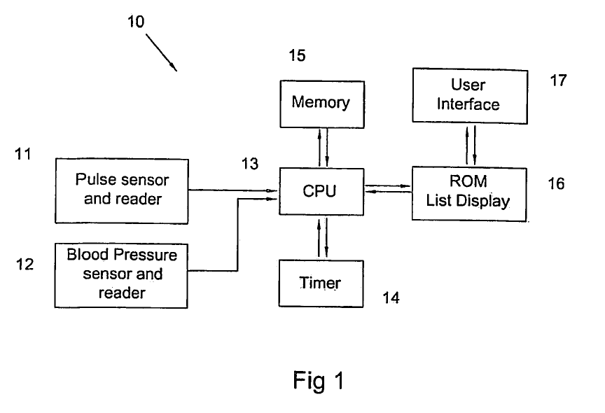

A pulse rate and rhythm monitoring device worn by a person that is exposed to one or more allergens in order to detect which one or more of the allergens produces an allergic reaction. The monitoring device is a wrist watch like device that is worn around the person's wrist, or can be worn anywhere on the body where there is a detectable pulse. The monitoring device 10 is shown in FIG. 1 and includes a pulse sensor and reader 11 that can detect a pulse from the person wearing the device 10, a blood pressure sensor and reader 12 that can detect the blood pressure of the person wearing the device 10, a CPU 13 that converts the pulse and blood pressure readings into data, a timer 14 to determine the time period between pulses and blood pressure readings, a memory 15 to store the data and the time periods between the sensor readings, a ROM list display 16 in which the list of potential allergens is displayed for the user to select and activate the monitoring device, and a user interface 17 that allows the user to select the item from the list in which the monitoring device is used to monitor for an allergic reaction. The device 10 can also include a means to upload and download data and lists of potential allergens to the device and to the computer software program. The means to upload and download could be a USB (universal serial bus) that uses a cable to connect the device 10 to a computer port, or the means could be a wireless device such as a Bluetooth or wifi connection through a wireless interface with the computer.

The device 10 includes within the memory and the controls a list of certain potential allergens such as various food items or cleaning products or outdoor environmental potential allergens in which the person is to be tested. This list of potential allergens can be downloaded from a software program to the monitoring device as needed. For example, the monitoring device 10 can have a list of food items such as corn, wheat, eggs and soy products that are known to cause allergic reactions in certain people. The device 10 allows for the person to select one or more of these items at a time so that the persons pulse can be monitored during the consumption of that item.

The monitoring device 10 includes a scroll button to scroll through the list of items to select one, a select button to select one of the items and to start the monitoring process, and a UBS or other connector that will allow for the monitoring device to be connected to a computer with the software that can download the data stored in the memory. Additional potential allergens can be downloaded into the device 10 for monitoring one at a time. Also, potential allergens on the list of the device can be removed when not needed by the computer program when the device 10 is connected to the computer or from the device 10 itself without the need of the computer software program. Each time the monitoring device 10 is connected to the computer program, the stored data can be erased after download in order to gather additional data for further monitoring.

When a person is exposed to a potential allergen, such as when she is eating a food item such as corn, the person will select from the list of items in the device the category “corn” and activate the device to start monitoring for the pulse rate, rhythm and amplitude and blood pressure for a period of time of around two hours after eating that item. By selecting one of the items from the list, the monitoring device will start monitoring for the various inputs (rhythm, rate, ect.) for the predetermined time period and store the data for later retrieval. The device will automatically stop monitoring after the two-hour (pre-selected time) period. However, the device is capable of setting the time period in which it will stop the monitoring process. Also, the time period for monitoring can be changed depending upon the circumstances.

Some people have severe allergic reactions that are clearly noticeable, while other reactions are more subtle and go unnoticed. That is why it is important to monitor the body for such unnoticed changes as the pulse rate and rhythm in order to detect for subtle allergic reactions. The prior art Pulse Rate Monitor of the Cohn invention and others like it take an average of the pulse rate for a certain time period and use that average to determine if an allergic reaction has taken place. This method can produce errors in that one person may have an allergic reaction that is slight and lasts for a short period of time. Thus, taking the average will not indicate that an allergic reaction has taken place. By plotting the various data obtained from the monitoring device of the present invention, the entire time period with the data points can be seen and the slight allergic reaction can be observed or identified.

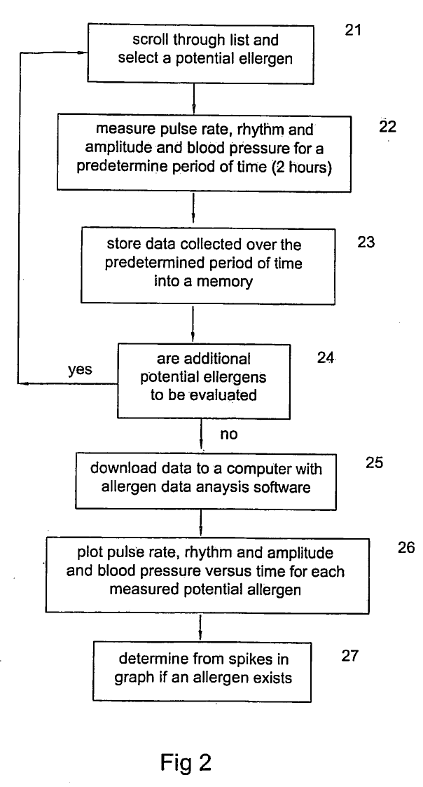

The operation of the device is represented by the flowchart in FIG. 2. to start the monitoring process, the user will attach the device 10 onto a part of the body where the pulse and blood pressure can be detected for the time period of the monitoring process. In this particular embodiment, the time period for each monitoring session is two hours. When the person is exposed to a potential allergen, such as eating corn for lunch, the person will scroll through the list of potential allergens on the device display and select that item, for example corn (step 21). When the person selects the item from the list, the monitoring device 10 starts the monitoring process by reading the pulse and blood pressure signals from the sensors. The device measures the pulse and blood pressure signals at the time intervals (step 22) and stores the data versus time in the memory (step 23). The individual pulses and blood pressure readings at each time period is stored in the memory. When the two hour period is expired, the device will stop the monitoring process. If an additional potential allergen is to be monitored, then the user will select from the list another item when the user is exposed to that item and the step 21 is started again.

If no additional allergens are to be monitored for, then the device is connected to a computer and the data collected from the monitoring process is downloaded into a software program (step 25) where the data is plotted versus time on a graph so that even small spikes can be observed (step 26). Because an average of pulse rate is not taken by the real time pulse rate, the graph of pulse rate versus time will show small spikes that would indicate an allergic reaction. If a slight allergic reaction was monitored, the prior art device of Cohn would not detect this because of the averaging process used. Also, because the monitoring device and process of the present invention also detects for pulse rhythm and amplitude and for blood pressure, other indicators of an allergic reaction besides the pulse rate can be used to evaluate a potential allergen.

Once an adequate amount of data has been collected by the device, the data is then downloaded into a software program where the data is analyzed to determine whether or not an allergic response is present. The program will compare the pulse rate, rhythm and amplitude to each of the food items or other potential allergen to detect for an allergic reaction. The device can then be reset and worn again to monitor for other food items or outdoor environmental conditions that can cause an allergic reaction.

The monitoring device of the present invention has preloaded list of the most common potential allergen items, but other items can also be entered individually by the user through an internal keyboard or from the software program that uses the stored data in which the device gathered.

For some allergies in some people, the allergic reaction can be eliminated by taken proper procedures to reintroduce the allergen into a person's diet in small increments to allow for the body to adapt to that allergen. Once a person has discovered an allergen, such as corn, that allergen can be eliminated in the person's diet for a period of time until that food item can be reintroduced after the body has adapted itself to that item. The food item is reintroduced into the diet on a rotational basis and in small quantities in order to determine whether this food item can be tolerated or will remain as a true allergen and therefore must be totally eliminated from the diet. By collecting the data from the monitoring device and storing the data on the software program, the reintroduction period for one or more allergens can be evaluated to determine if one or more are true allergens.

The use of the monitoring device would be as follows. A person would connect the monitoring device to a computer with preloaded software and select certain potential allergens, such as food items, in which the person will be exposed to during the initial monitoring period. The person would then place the monitoring device on the body where the pulse is detected by the device. When the person is exposed to a certain potential allergen, such as starting to eat corn, the person will select that item on the monitoring device and activate the key that indicates to the monitoring device that corn is being consumed. The monitoring device will then proceed to monitor the person's pulse rate, rhythm and amplitude and blood pressure for a predetermined period of time such as two hours. A person will probably eat other items as well other than the corn. So, the person can select other items listed on the monitoring device that are also being consumed along with the corn for monitoring. If an allergic reaction is detected, then future monitoring can be such that one or more of the items in the original lists can be eliminated in order to detect which of the items or item produced the allergic reaction. By monitoring several times and downloading the data collected from the several monitoring events, one or more allergens can be identified from a list of many that the person has been exposed to. Through a process of elimination, the real allergen can be identified.

A person may have a belief that one certain item may be an allergen. The person can then be exposed to that potential allergen along with other items while monitoring the body for the various signals to detect for an allergic reaction. In future monitoring periods, that potential allergen can be eliminated in order to confirm that is was indeed an allergen.

Another benefit of the monitoring device of the present invention is that the person wearing the device does not have to understand how it works or how it detects if an allergen is identified. All that the person has to do is to select the item that she is exposed to so that the monitoring device will monitor the pulse for the pre-selected period of time. When the person selects the item from the preloaded list of items, such as corn, the monitoring device will start the monitoring process. When the monitoring period has ended, such as one day or one week, the monitoring device is brought to a person who can download the data and interpret that data to detect for one or more allergens. Thus, the monitoring device of the present invention can be used on an infant or an elderly person who may not know how the device works other than being able to select the certain items from the list and the time of exposure.

The software program that retrieves the data from the monitoring device will plot the pulse rate, rhythm and other data points on a graph for each of the items that were monitored. From the graph of the data points, a person skilled in interpreting the data can observe if one of the items produces the results that would indicate an allergic reaction has occurred. One advantage of the monitoring device of the present invention is that the data represents real time events and not just an average for a time period so that even slight changes in the pulse rate, rhythm and others can be seen that would indicate even a slight allergic reaction. Also, monitoring for the pulse rate as well as the rhythm and amplitude and the blood pressure provided additional indicators of an allergic reaction that will not show on only the pulse rate.

I claim the following:

1. A wearable pulse rate monitoring device comprising:
A display with a list of potential allergens;
Means to select one of the potential allergens from the list;
A pulse sensor to detect a pulse from a person wearing the monitoring device;
A processor to convert the pulse sensor signals into real time data of the pulse signals versus time; and,
A memory to store the real time data of the pulse signals versus time.

2. The wearable pulse rate monitoring device of claim 1, and further comprising:
Communication means to transmit data between the monitoring device and a computer.

3. The wearable pulse rate monitoring device of claim 1, and further comprising:
The processor includes means to convert the pulse sensor signal into pulse rate, pulse rhythm and pulse magnitude versus time for a predetermined period of time.

4. The wearable pulse rate monitoring device of claim 1, and further comprising:
The means to select one of the, potential allergens is also the means to activate the monitoring device.

5. The wearable pulse rate monitoring device of claim 1, and further comprising:
A blood pressure sensor to detect for a blood pressure.

6. The wearable pulse rate monitoring device of claim 1, and further comprising:
The communication means to transmit data is a USB or a wireless interface.

7. A process for monitoring a person for pulse rate to determine the presence of an allergen, the process comprising the step of:
Exposing the person to a potential allergen form a list of potential allergens;
Monitoring the person's pulse rate versus time in real time for a pre-selected period of time;
Storing the pulse rate versus time in a memory; and,
Plotting the pulse rate versus time for the selected potential allergen on a graph representing pulse versus time for the pre-selected period of time.

8. The process for monitoring a person for pulse rate of claim 7, and further comprising the step of:
Monitoring the person's pulse rhythm versus time.

9. The process for monitoring a person for pulse rate of claim 8, and further comprising the step of:
Monitoring the person's pulse amplitude versus time.

10. The process for monitoring a person for pulse rate of claim 7, and further comprising the steps of:
Downloading the collected data from the wearable pulse sensor device into a computer program to plot the data versus time; and,
Uploading to the wearable pulse sensor device additional potential allergens to be monitored for.

11. The process for monitoring a person for pulse rate of claim 7, and further comprising the step of:
Monitoring the person's blood pressure versus time.
